(19) United States
(12) Patent Application Publication
(10) Pub. No.: US 2021/0018479 A1
(43) Pub. Date: Jan. 21, 2021

(54) ODOR SENSING APPARATUS, ODOR DETECTION METHOD, AND COMPUTER-READABLE RECORDING MEDIUM

(71) Applicant: NEC CORPORATION, Tokyo (JP)

(72) Inventors: Junko WATANABE, Tokyo (JP); Riki ETO, Tokyo (JP); Hidetaka HANE, Tokyo (JP); Shigeo KIMURA, Tokyo (JP); Shintarou TSUCHIYA, Tokyo (JP)

(73) Assignee: NEC CORPORATION, Tokyo (JP)

(21) Appl. No.: 16/969,621

(22) PCT Filed: Feb. 22, 2019
(86) PCT No.: PCT/JP2019/006893

§ 371 Date: Aug. 13, 2020

(30) Foreign Application Priority Data
Feb. 26, 2018 (JP) 2018-032076

(51) Int. Cl. G01N 33/00; G01N 27/02

(52) U.S. Cl. CPC G01N 33/0031; G01N 27/02

(57) ABSTRACT

An odor detection apparatus 100 includes a first odor sensor 10 provided with a sensitive membrane, a second odor sensor 20 provided with an identical sensitive membrane, and a control device 30. The control device 30 includes a sensor data acquisition unit 31 that acquires first sensor data output by the first odor sensor 10 and second sensor data output by the second odor sensor, a calculation processing unit 32 that calculates a difference between the first sensor data and the second sensor data, and a determination unit 33 that determines, when the sensitive membrane of one of the odor sensors is in a steady state, whether the sensitive membrane of the other odor sensor is in a steady state, based on the difference.

### TECHNICAL FIELD

The present invention relates to an odor detection apparatus and an odor detection method that are for detecting an odor from a substance in the atmosphere, and to a computer-readable recording medium for realizing the apparatus and method.

### BACKGROUND ART

Heretofore, odor sensors have been used in order to detect specific odors (e.g., refer to Patent Documents 1 and 2). An odor sensor detects a specific odor by detecting molecules that cause the specific odor (hereinafter, “odor molecules”) with a sensitive membrane. Also, a metal oxide film and an organic semiconductor thin film are given as examples of a sensitive membrane. With such a sensitive membrane, conductivity changes when specific odor molecules adhere thereto, thus enabling specific chemical substances to be detected.

Also, with a conventional odor sensor, the odor molecules that can be detected are fixed, and thus the odors that are detected are also fixed, although in recent years, general-purpose odor sensors that are able to detect various types of odors with one sensor have been developed. For example, Non-Patent Literature 1 discloses a membrane-type surface stress sensor (MSS) as a general-purpose odor sensor.

An MSS is usually constituted by two or more sensor elements. Each sensor element includes a circular portion provided with a sensitive membrane, a frame surrounding the circular portion, and a plurality of bridges for coupling the circular portion to the frame. A piezoresistive element is embedded in each bridge. In such a configuration, the circular portion deforms due to stress occurring in the sensitive membrane when odor molecules stick to the sensitive membrane, resulting in stress being applied to the bridges. As a result, the electrical resistance of the piezoresistive elements embedded in the bridges changes greatly, thus enabling the odor to be detected from the resistance value.

Also, with an MSS, the material of the sensitive membrane differs for every sensor element, but this does not necessarily mean that the substance that sticks to each sensor element is fixed to one type. The material of the sensitive membrane of each sensor element is configured such that the pattern of the output data of the entire MSS that is obtained by compositing the output data of the respective sensor elements differs according to the odor, that is, the set of odor molecules that cause the odor. Thus, with an MSS, it becomes possible to detect multiple types of odors, by learning output patterns and creating analyzers, for every odor that serves as a detection target, through machine learning in advance.

Note that there is also a technique that involves using a plurality of odor sensors having different characteristics including odor sensors other than an MSS, performing analysis through machine learning by combining data from the respective odor sensors, and generating an odor analyzer for every target.

### LIST OF RELATED ART DOCUMENTS

### Patent Document

Patent Document 1: Japanese Patent No. 6121014

Patent Document 2: Japanese Patent No. 5582803

### Non-Patent Document

Non-Patent Document 1: MSS alliance launched to set de facto standard for odor-sensing systems—aiming to establish basic elements of MSS technology towards practical use—[online], Sep. 29, 2015, NEC Corp., [viewed on Sep. 1, 2015], Internet <URL: http://jpn.nec.com/press/201509/20150929_01.html>

### SUMMARY OF INVENTION

### Problems to be Solved by the Invention

Incidentally, with such an odor sensor provided with a sensitive membrane, processing for eliminating (purging) the odor molecules adhering to the sensitive membrane may be performed before performing detection, in order to place the sensitive membrane in a stationary state, so as to maintain the detection accuracy. This odor molecule elimination processing (purge processing) is particularly important in a general-purpose odor sensor that detects a plurality of types of odors.

However, with conventional odor sensors, there was no way of knowing that the elimination processing had been sufficiently performed for the sensitive membrane to be in a steady state, and the user assumed that the elimination processing was completed after a given time period on the basis of experience and test values. Detection accuracy may thus decrease since the elimination processing may be insufficient. Also, there may be cases where the elimination processing is performed for an unnecessarily long time, and work efficiency decreases.

An example object of the invention is to provide an odor sensing apparatus, an odor detection method and a computer-readable recording medium that solve the above problems, and are capable of determining whether a sensitive membrane is in a steady state in elimination processing on an odor sensor.

### Means for Solving the Problems

An odor detection apparatus according to an example aspect of the invention includes:

a first odor sensor provided with a sensitive membrane;

a second odor sensor provided with an identical sensitive membrane to the sensitive membrane of the first odor sensor; and

a control device,

the control device including:

a sensor data acquisition unit configured to acquire first sensor data output by the first odor sensor and second sensor data output by the second odor sensor;

a calculation processing unit configured to calculate a difference between the first sensor data and the second sensor data; and

a determination unit configured to determine, when the sensitive membrane of one of the odor sensors is in a steady state, whether the sensitive membrane of the other odor sensor is in a steady state, based on the difference.

Also, an odor detection method according to an example aspect of the invention is for detecting an odor, using a first odor sensor provided with a sensitive membrane and a second odor sensor provided with an identical sensitive membrane to the sensitive membrane of the first odor sensor, the method including:

(a) a step of acquiring first sensor data output by the first odor sensor and second sensor data output by the second odor sensor;

(b) a step of calculating a difference between the first sensor data and the second sensor data; and

(c) a step of determining, when the sensitive membrane of one of the odor sensors is in a steady state, whether the sensitive membrane of the other odor sensor is in a steady state, based on the difference.

Furthermore, a computer-readable recording medium according to an example aspect of the invention includes a program recorded thereon, in an odor sensing apparatus including a first odor sensor provided with a sensitive membrane, a second odor sensor provided with an identical sensitive membrane to the sensitive membrane of the first odor sensor and a computer, the program including instructions that cause the computer to carry out:

(a) a step of acquiring first sensor data output by the first odor sensor and second sensor data output by the second odor sensor;

(b) a step of calculating a difference between the first sensor data and the second sensor data; and

(c) a step of determining, when the sensitive membrane of one of the odor sensors is in a steady state, whether the sensitive membrane of the other odor sensor is in a steady state, based on the difference.

### Advantageous Effects of the Invention

As described above, according to the invention, it can be determined whether a sensitive membrane in a steady state in elimination processing on an odor sensor.

### BRIEF DESCRIPTION OF THE DRAWINGS

FIG. 1 is a block diagram showing a schematic configuration of an odor detection apparatus in a first example embodiment of the invention.

FIG. 2 is an external view showing an external appearance of the odor detection apparatus in the first example embodiment of the invention.

FIG. 3 is a block diagram showing a detailed configuration of the odor detection apparatus in the first example embodiment of the invention.

FIG. 4 is a diagram showing an example of sensor data that is output by an odor sensor in the first example embodiment of the invention.

FIG. 5 is a diagram showing an example of sensor data that is output when an odor sensor is in a steady state in the first example embodiment of the invention.

FIG. 6 shows an example of information that is displayed on a screen of the odor detection apparatus in the first example embodiment of the invention.

FIG. 7 is a flowchart showing operations of the odor detection apparatus in the first example embodiment of the invention.

FIG. 8 is a block diagram showing a schematic configuration of a first variation of the odor detection apparatus in the first example embodiment of the invention.

FIG. 9 is a diagram showing a schematic configuration of a second variation of the odor detection apparatus in the first example embodiment of the invention.

FIG. 10 is a block diagram showing a detailed configuration of an odor detection apparatus in a second example embodiment of the invention.

FIG. 11 is a flowchart showing operations of the odor detection apparatus in the second example embodiment of the invention.

FIG. 12 is a block diagram showing an example of a computer that realizes the odor detection apparatus in the first and second example embodiments of the invention.

### EXAMPLE EMBODIMENTS

### First Example Embodiment

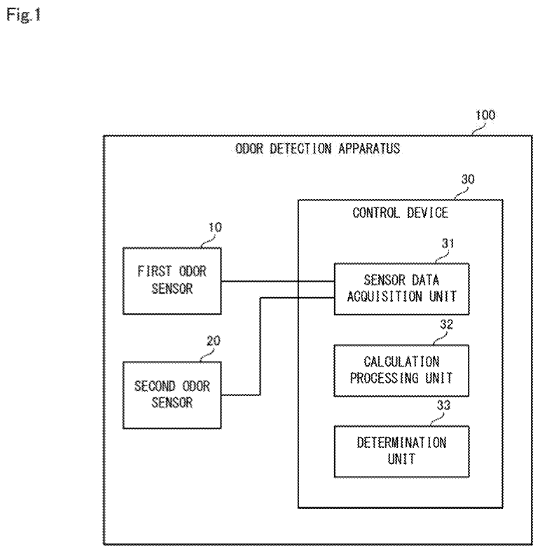

Hereinafter, an odor sensing apparatus, an odor detection method and a program in a first example embodiment of the invention will be described, with reference to FIGS. 1 to 8.

### Apparatus Configuration

Initially, a schematic configuration of the odor detection apparatus in the first example embodiment will be described using FIG. 1. FIG. 1 is a block diagram showing a schematic configuration of the odor detection apparatus in the first example embodiment of the invention.

The odor detection apparatus 100 in the first example embodiment shown in FIG. 1 is an apparatus that detects odors using odor sensors. As shown in FIG. 1, the odor detection apparatus 100 in the first example embodiment is provided with a first odor sensor 10, a second odor sensor 20, and a control device 30.

The first odor sensor 10 is an odor sensor provided with a sensitive membrane. Also, the second odor sensor 20 is an odor sensor provided with an identical sensitive membrane to the sensitive membrane of the first odor sensor 10.

Also, in this example embodiment, “odor” includes not only odors that people experience with their sense of smell but also odors that people cannot experience with their sense of smell. Also, solids and liquids that emit odors, and, furthermore, the contraction of diseases are also included as targets for odor detection, in addition to gases containing odor molecules. Furthermore, gases containing odor molecules include the expiration of organisms, gas molecules produced from excrement, and indoor and outdoor ambient air. Also, foodstuffs, degraded structures and foodstuffs are given as solids that emit odors. Bodily fluids of organisms, sweat, drinking water and alcohol are given as liquids that emit odors. Also, targets for “odor” detection include substances that cause odors, and, furthermore, the degree of ripeness of fruit that likewise causes odors, and the degree of degradation of structures that likewise causes odors.

Also, as shown in FIG. 1, the control device 30 is provided with a sensor data acquisition unit 31, a calculation processing unit 32, and a determination unit 33. The sensor data acquisition unit 31 acquires first sensor data output by the first odor sensor and second sensor data output by the second odor sensor.

The calculation processing unit 32 calculates the difference between the first sensor data and the second sensor data. The determination unit 33 determines, when the sensitive membrane of one of the odor sensors is in a steady state, whether the sensitive membrane of the other odor sensor is in a steady state, based on the difference.

In this way, in this example embodiment, two odor sensors are provided, thus enabling odor detection to be performed by one odor sensor, and the other odor sensor to be placed in a steady state. Thus, according to this example embodiment, by using the sensor data from the odor sensor that is in a steady state, it can be determined whether the sensitive membrane of the odor sensor that performed odor detection has achieved a steady state due to elimination processing.

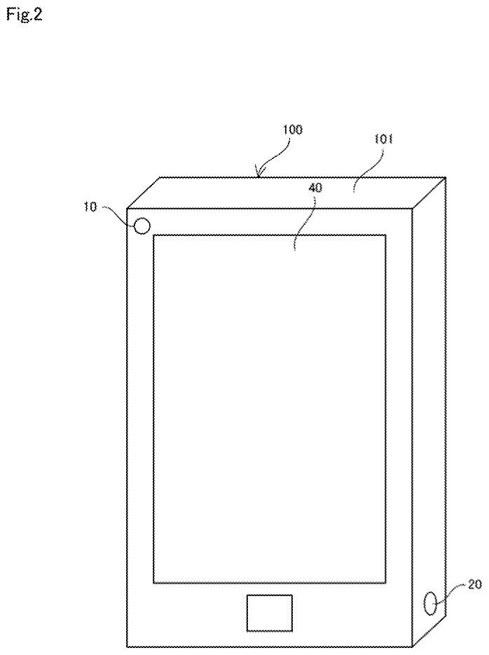

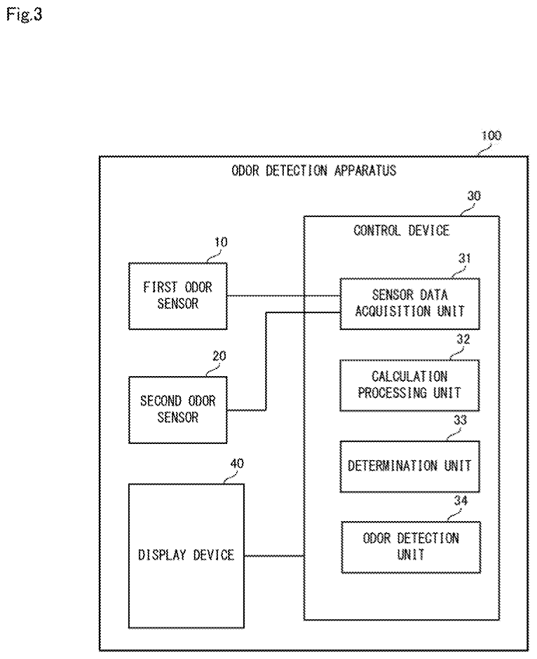

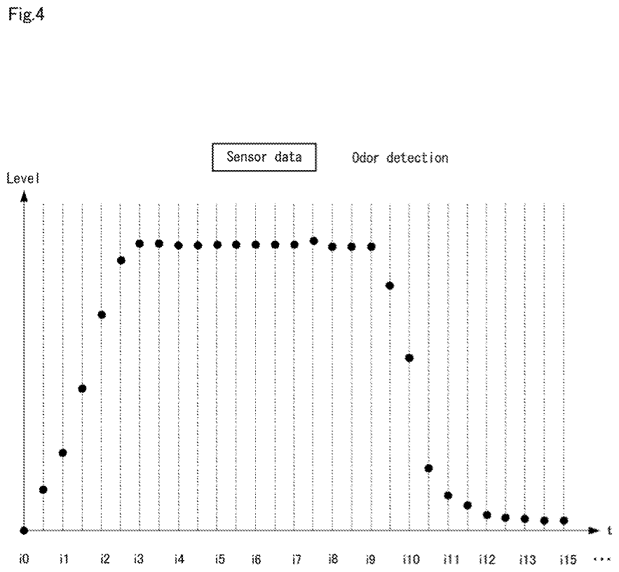

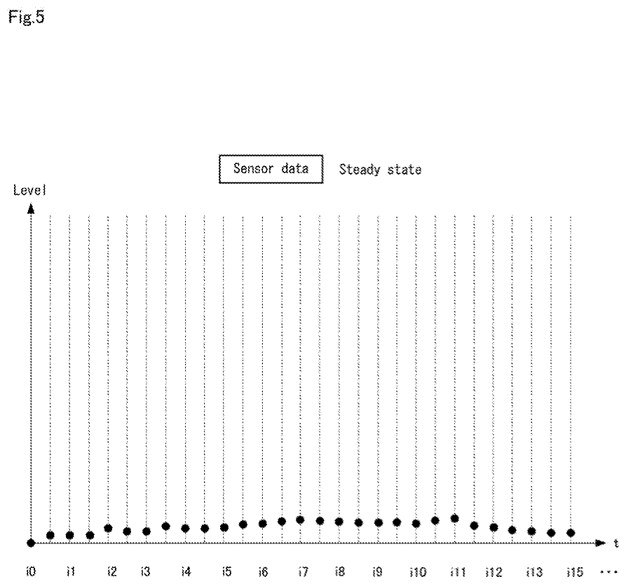

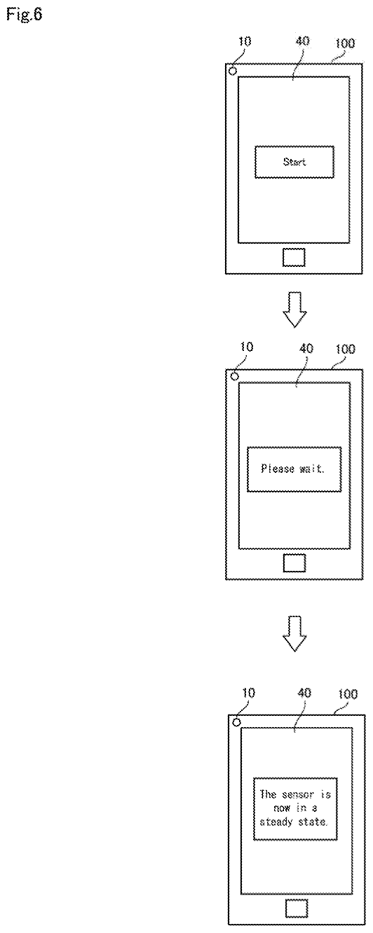

Here, the configuration of the odor detection apparatus 100 in the first example embodiment will be described in more specifically using FIGS. 2 to 5. FIG. 2 is an external diagram showing an external appearance of the odor detection apparatus in the first example embodiment of the invention. FIG. 3 is a block diagram showing a detailed configuration of the odor detection apparatus in the first example embodiment of the invention. FIG. 4 is a diagram showing an example of sensor data that is output by an odor sensor in the first example embodiment of the invention. FIG. 5 is a diagram showing an example of sensor data that is output when an odor sensor is in a steady state in the first example embodiment of the invention. FIG. 6 shows an example of information that is displayed on a screen of the odor detection apparatus in the first example embodiment of the invention.

First, as shown in FIG. 2, in the first example embodiment, the odor detection apparatus 100 has a portable configuration. The first odor sensor 10 and the second odor sensor 20 shown in FIG. 1 are attached to different parts of a casing 101 constituting the odor detection apparatus 100. The control device 30 is housed in the casing 101. In FIG. 2, reference numeral 40 denotes a display device which will be discussed later.

Also, as shown in FIG. 3, in the first example embodiment, the odor detection apparatus 100 is provided with the display device 40, in addition to the first odor sensor 10, the second odor sensor 20, and the control device 30. Furthermore, the control device 30 is provided with an odor detection unit 34, in addition to the sensor data acquisition unit 31, the calculation processing unit 32, and the determination unit 33.

The odor detection unit 34 detects odors, based on the sensor data from one of the first odor sensor and the second odor sensor. Specifically, the odor detection unit 34 detects odors, using the sensor data of an odor sensor that touches or is close to the liquid, gas or solid that emits odor molecules. Also, the odor detection unit 34 displays a detection result on a screen of the display device 40.

Here, processing by the odor detection unit 34 in the case where an odor sensor provided with a sensitive membrane is used as the first odor sensor 10 and the second odor sensor 20 will be described. First, when the odor sensor provided with a sensitive membrane contacts a sample gas containing odor molecules, the sensor data shown in FIG. 4 is output as sensor data. Also, the odor sensor provided with a sensitive membrane outputs the sensor data shown in FIG. 5, for example, when in a steady state.

Examples of sensor data output by one of the sensor elements constituting an odor sensor provided with a sensitive membrane are shown in FIGS. 4 and 5. Also, the first odor sensor 10 and the second odor sensor 20 output sensor data at a set sampling rate. Note that i0, i1, i2 and so on that are shown in FIGS. 4 and 5 are indexes, a description of which will be given later. Also, in the indexes, “i” indicates output from a specific sensor element. In the case of output from a different sensor element, a letter other than “i” is allocated.

In the case where such an odor sensor provided with a sensitive membrane is used as the first odor sensor 10 and the second odor sensor 20, the odor detection unit 34 first selects an analyzer corresponding to the odor serving as a detection target from among analyzers that have been prepared in advance.

The analyzer is an analysis model created in advance by machine learning. Analysis models are created by machine learning the features of sensor data, with a support vector machine, using sensor data output by the odor sensor when there is a reaction to test odors as learning data, and using data specifying test odors as training data, for example.

Also, in this example embodiment, machine learning is performed utilizing the sparsity of sensor data, for example. In other words, the chemical behavior of an odor that serves as an analysis target and sensor elements and the physical characteristics of the odor sensor are aggregated and appear in specific portions within the sensor data. In the example in FIG. 4, such behavior and characteristics appear in the rising portion of sensor data, the flat upper portion of the waveform, and the like. Accordingly, an appropriate analysis model can also be built with machine learning that uses only a specific portion of the sensor data. Furthermore, the analysis accuracy can also be determined by the amount of data that is used in machine learning.

In this example embodiment, machine learning may thus be performed by extracting only an effective portion of sensor data serving as learning data. By using a technology called feature selection, for example, the portion effective for learning and analysis can be specified and extracted from sensor data serving as learning data. Also, the portion that is extracted at this time is specified by the indexes shown in FIG. 4.

The odor detection unit 34, upon an analyzer being selected, applies sensor data to the selected analyzer and detects an odor. Specifically, the odor detection unit 34 specifies the concentration of odor molecules from the output result of the analyzer, and estimates the stepwise level of the odor according to the specified concentration. The odor detection unit 34 then displays the estimated level of the odor on the screen of the display device 40.

The sensor data acquisition unit 31 acquires the sensor data that is output by the first odor sensor 10 and the sensor data that is output by the second odor sensor 20 whenever this sensor data is output, and passes the acquired sensor data to the calculation processing unit 32. The calculation processing unit 32, upon receiving the first sensor data and the second sensor data, calculates the difference between the output values of the first and second sensor data output in the same period, and passes the calculated difference to the determination unit 33.

Also, in the first example embodiment, the first odor sensor 10 is used in detection of odors and the second odor sensor 20 is used in specification of a steady state. Accordingly, in the first example embodiment, the determination unit 33 determines whether the sensitive membrane of the first odor sensor 10 is in a steady state, based on the difference between the first sensor data and the second sensor data. Specifically, the determination unit 33 determines that the sensitive membrane of the first odor sensor 10 is in a steady state, on condition that the difference is less than or equal to a threshold value, and that state continues for a set time period, for example.

Also, upon the determination processing by the determination unit 33 being started, the control device 30 presents a message on the screen of the display device 40, as shown in FIG. 6. The user can thereby know whether the sensitive membrane of the first odor sensor 10 is in a steady state.

### Apparatus Operations

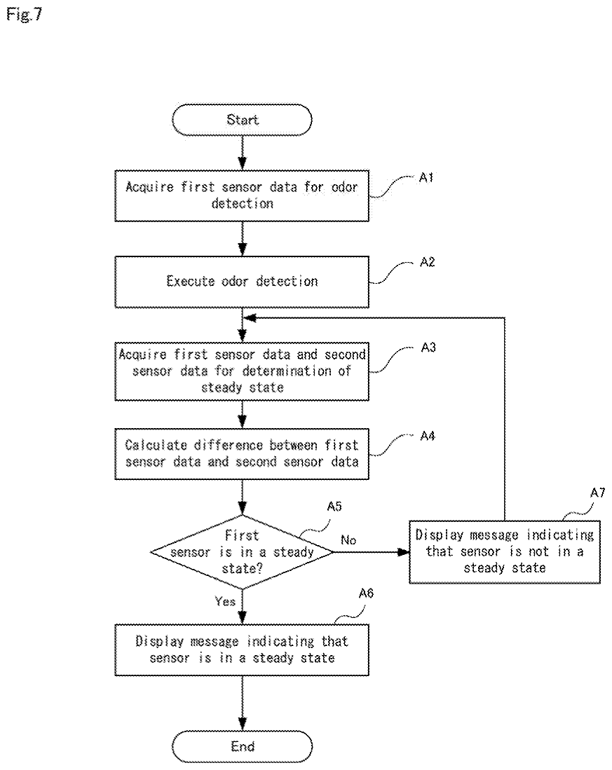

Next, operations of the server apparatus 100 in the first example embodiment will be described using FIG. 7. FIG. 7 is a flowchart showing operations of the odor detection apparatus in the first example embodiment of the invention. In the following description, FIG. 1 will be referred to as appropriate. Also, in the first example embodiment, the odor detection method is implemented by operating the odor detection apparatus 100. Therefore, the following description of the operations of the odor sensing apparatus 100 will be given in place of a description of the odor detection method in the first example embodiment.

Initially, as shown in FIG. 7, in the control device 30, the sensor data acquisition unit 31, in order to perform odor detection, acquires a set number of samplings of sensor data output by the first odor sensor 10 (step A1). Next, the odor detection unit 34 detects an odor by applying the sensor data acquired at step A1 to the analyzer (step A2).

Next, when step A2 has ended, the sensor data acquisition unit 31, for determination of the steady state of the first odor sensor, acquires the first sensor data from the first odor sensor 10 and acquires the second sensor data from the second odor sensor 20 (step A3).

Next, the calculation processing unit 32 calculates the difference between the first sensor data acquired in step A3 and the second sensor data likewise acquired in step A3 (step A4). Also, in step A4, the calculation processing unit 32 passes the calculated difference to the determination unit 33.

Next, the determination unit 33 determines whether the sensitive membrane of the first odor sensor 10 is in a steady state, based on the difference between the first sensor data and the second sensor data (step A5).

If the determination result of step A5 indicates that the sensitive membrane is in a steady state, the determination unit 33 presents a message indicating that the sensitive membrane is in a steady state on the screen of the display device 40 (step A6: refer to lower level of FIG. 6). On the other hand, if the determination result of step A4 indicates that the sensitive membrane is not in a steady state, the determination unit 33 presents a message indicating that the sensitive membrane is not yet in a steady state on the screen of the display device 40 (step A7: refer to middle level of FIG. 6).

In this way, according to the first example embodiment, it can be determined whether the sensitive membrane of the odor sensor that performed odor detection has achieved a steady state due to elimination processing. According to the first example embodiment, highly accurate odor detection can be performed, using odor sensors provided with a sensitive membrane.

### Program

The program in the first example embodiment need only be a program that causes a computer to execute steps A1 to A7 shown in FIG. 7. The odor detection apparatus 100 and the odor detection method in the first example embodiment can be realized, by this program being installed on a computer and executed. In this case, a processor of the computer performs processing while functioning as the sensor data acquisition unit 31, the calculation processing unit 32, the determination unit 33 and the odor detection unit 34 in the control device 30. Also, a computer constituting the control device 30 of the odor detection apparatus 100 is given as the computer.

### First Variation

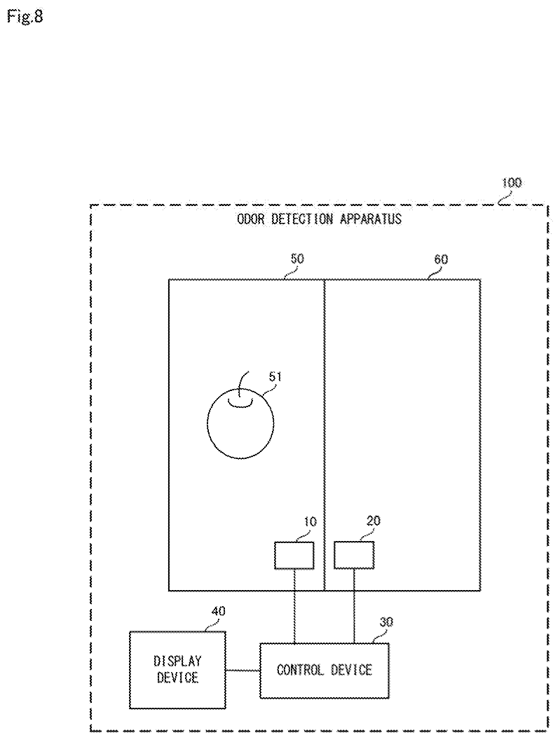

Next, a first variation of the odor detection apparatus 100 in the first example embodiment will be described using FIG. 8. FIG. 8 is a diagram showing a schematic configuration of the first variation of the odor detection apparatus in the first example embodiment of the invention.

As shown in FIG. 8, in this variation, the odor detection apparatus 100 is provided with two chambers, namely, a chamber 50 and a chamber 60. Also, the first odor sensor 10 is disposed inside the chamber 50, and the second odor sensor 20 is disposed inside the chamber 60. The first odor sensor 10 and the second odor sensor 20 are isolated from each other.

In the example in FIG. 8, fruit 51 has been placed in the chamber 50, whereas nothing except ambient air is in the chamber 60. With this first variation shown in FIG. 8, it can thus similarly be determined whether the sensitive membrane of the first odor sensor 10 is in a steady state, by using the sensor data from the second odor sensor 20.

### Second Variation

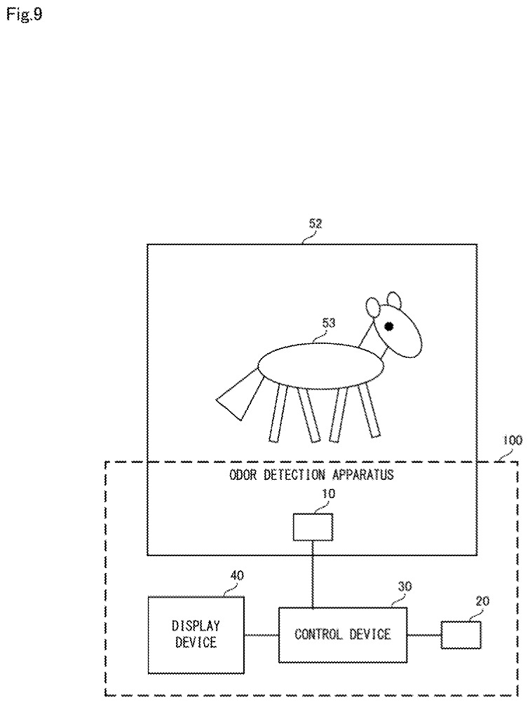

Next, a second variation of the odor detection apparatus 100 in the first example embodiment will be described using FIG. 9. FIG. 9 is a diagram showing a schematic configuration of the second variation of the odor detection apparatus in the first example embodiment of the invention.

As shown in FIG. 9, in this second variation, the odor detection apparatus 100 is attached to a building 52 that holds livestock such as a horse stable. The first odor sensor 10 is disposed inside the building 52, and the second odor sensor 20 is disposed outside the building 52. In this second variation, the first odor sensor 10 and the second odor sensor 20 are similarly isolated from each other.

In the example in FIG. 9, a horse 53 is kept inside the building 52. The odor of the horse 53 is thus detected by the first odor sensor 10. Also, the second odor sensor 20 is exposed to the ambient air and is in a steady state. Accordingly, with this second variation shown in FIG. 9, it can similarly be determined whether the sensitive membrane of the first odor sensor 10 is in a steady state, by using the sensor data from the second odor sensor 20.

### Second Example Embodiment

Hereinafter, an odor detection apparatus, an odor detection method and a program in a second example embodiment of the invention will be described, with reference to FIGS. 9 to 11.

### Apparatus Configuration

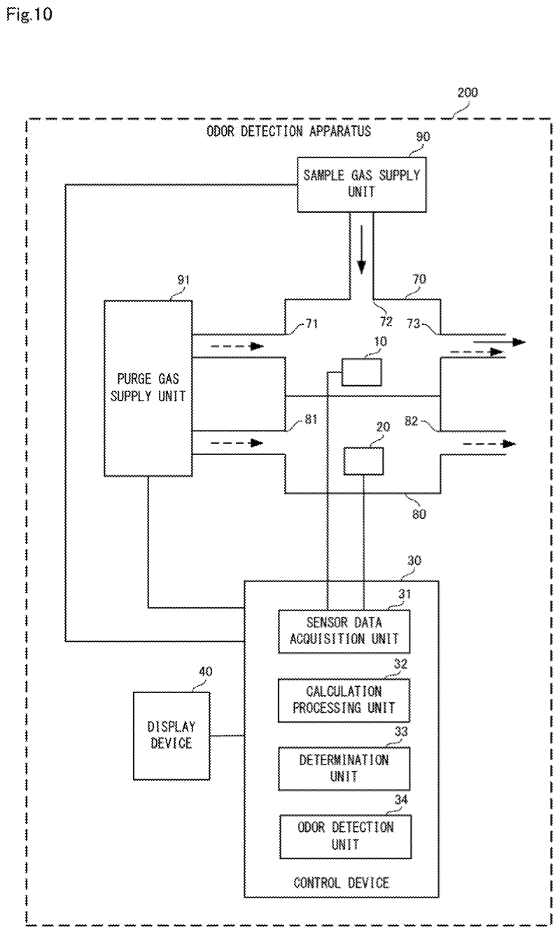

Initially, the configuration of the odor detection apparatus in the second example embodiment will be described using FIG. 10. FIG. 10 is a block diagram showing a detailed configuration of the odor detection apparatus in the second example embodiment of the invention.

As shown in FIG. 10, an odor detection apparatus 200 in the second example embodiment is also provided with a first odor sensor 10, a second odor sensor 20 and a control device 30, similarly to the odor detection apparatus 100 in the first example embodiment shown in FIG. 3, but differs in the following respects. The following description will focus on the differences from the first example embodiment.

As shown in FIG. 10, in the second example embodiment, the odor detection apparatus 200 is provided with a first chamber 70, a second chamber 80, a sample gas supply unit 90 and a purge gas supply unit 91, in addition to the configuration shown in FIG. 3. Also, the first odor sensor 10 is disposed in the first chamber 70, and the second odor sensor 20 is disposed in the second chamber 80.

The sample gas supply unit 90 supplies a sample gas containing odor molecules to the first chamber 70. Specifically, the sample gas supply unit 90 has a pump for supplying the sample gas and a valve for starting or stopping supply of the sample gas. Also, the pump and valve of the sample gas supply unit 90 are controlled by the control device 30.

The purge gas supply unit 91 supplies a purge gas (e.g., inert gas such as nitrogen gas) for eliminating odor molecules from the sensitive membrane to the first chamber 70 and the second chamber 80. Specifically, the purge gas supply unit 91 has a pump for supplying the purge gas and a valve for starting or stopping supply of the purge gas. Also, the pump and valve of the purge gas supply unit 91 are controlled by the control device 30.

Also, an inlet port 71 for introducing the purge gas, an inlet port 72 for introducing the sample gas, and an exhaust port 73 for exhausting the purge gas and the sample gas are provided in the first chamber 70. In addition, an inlet port 81 for introducing the purge gas and an exhaust port 82 for exhausting the purge gas are provided in the second chamber 80.

Also, in the second example embodiment, the control device 30 supplies the sample gas to the first chamber 70 with the sample gas supply unit 90, in the case of performing odor detection with the first odor sensor 10. In the first chamber 70, the sample gas is thereby introduced through the inlet port 72 and exhausted through the exhaust port 73. Also, the control device 30 causes the odor detection unit 34 to perform odor detection while continuing supply of the sample gas.

On the other hand, after odor detection has ended and supply of the sample gas by the sample gas supply unit 90 has ended, the control device 30 causes the purge gas supply unit 91 to supply the purge gas to the first chamber 70 and the second chamber 80. In the first chamber 70, the purge gas is thereby introduced through the inlet port 71 and exhausted through the exhaust port 73. Also, in the second chamber 80, the purge gas is introduced through the inlet port 81 and exhausted through the exhaust port 82.

The control device 30 then executes sensor data acquisition with the sensor data acquisition unit 31, difference calculation with the calculation processing unit 32, and determination with the determination unit 33, while continuing supply of the purge gas.

### Apparatus Operations

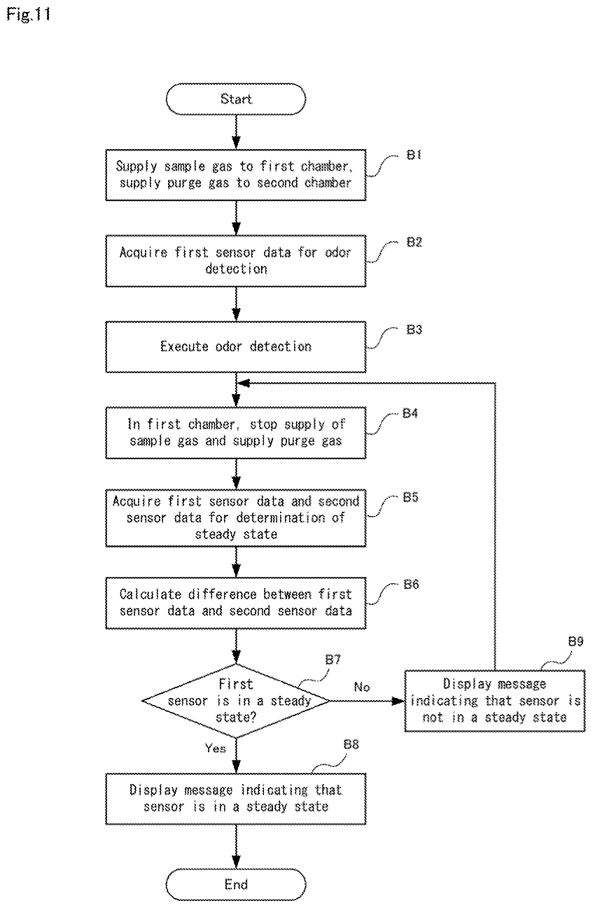

Next, operations of the odor detection apparatus 200 in the second example embodiment will be described using FIG. 11. FIG. 11 is a flowchart showing operations of the odor detection apparatus in the second example embodiment of the invention. In the following description, FIG. 9 will be referred to as appropriate. Also, in the second example embodiment, the odor detection method is implemented by operating the odor detection apparatus 200. Therefore, the following description of the operations of the odor sensing apparatus 200 will be given in place of a description of the odor detection method in the second example embodiment.

Initially, as shown in FIG. 11, the control device 30, in order to perform odor detection with the first odor sensor 10, operates the sample gas supply unit 90 to supply the sample gas to the first chamber 70 (step B1). The control device 30 also operates the purge gas supply unit 91 to supply the purge gas to the second chamber 80.

Next, in the control device 30, the sensor data acquisition unit 31, in order to perform odor detection, acquires a set number of samplings of sensor data output by the first odor sensor 10 (step B2). Next, the odor detection unit 34 detects an odor by applying the sensor data acquired in step B1 to the analyzer (step B3).

Next, when step B3 has ended, the control device 30 stops operation of the sample gas supply unit 90 to stop supply of the sample gas to the first chamber 70. The control device 30 then also supplies the purge gas to the first chamber 70 with the purge gas supply unit 91 (step B4).

Next, the sensor data acquisition unit 31, for determination of the steady state of the first odor sensor, acquires the first sensor data from the first odor sensor 10 and acquires the second sensor data from the second odor sensor 20 (step B5).

Next, the calculation processing unit 32 calculates the difference between the first sensor data acquired in step B5 and the second sensor data similarly acquired in step B5 (step B6). Also, in step B6, the calculation processing unit 32 passes the calculated difference to the determination unit 33.

Next, the determination unit 33 determines whether the sensitive membrane of the first odor sensor 10 is in a steady state, based on the difference between the first sensor data and the second sensor data (step B7).

If the determination result of step B7 indicates that the sensitive membrane is in a steady state, the determination unit 33 presents a message indicating that the sensitive membrane is in a steady state on the screen of the display device 40 (step B8: refer to lower level of FIG. 6). On the other hand, if the determination result of step A4 indicates that the sensitive membrane is not in a steady state, the determination unit 33 presents a message indicating that the sensitive membrane is not yet in a steady state on the screen of the display device 40 (step B9: refer to middle level of FIG. 6).

In this way, in the second example embodiment, it can similarly be determined whether the sensitive membrane of the odor sensor that performed odor detection has achieved a steady state due to elimination processing. Also, in the case of the second example embodiment, highly accurate odor detection can similarly be performed, using odor sensors provided with a sensitive membrane. Also, in the second example embodiment, since the purge gas is supplied to the odor sensor that performed odor detection, odor molecules can be reliably eliminated in a short time, compared with the first example embodiment.

### Program

The program in the second example embodiment need only be a program that causes a computer to execute steps B1 to B8 shown in FIG. 11. The odor detection apparatus 200 and the odor detection method in the second example embodiment can be realized, by this program being installed on a computer and executed. In this case, a processor of the computer performs processing while functioning as the sensor data acquisition unit 31, the calculation processing unit 32, the determination unit 33 and the odor detection unit 34 in the control device 30. Also, a computer constituting the control device 30 of the odor detection apparatus 200 is given as the computer.

### Physical Configuration

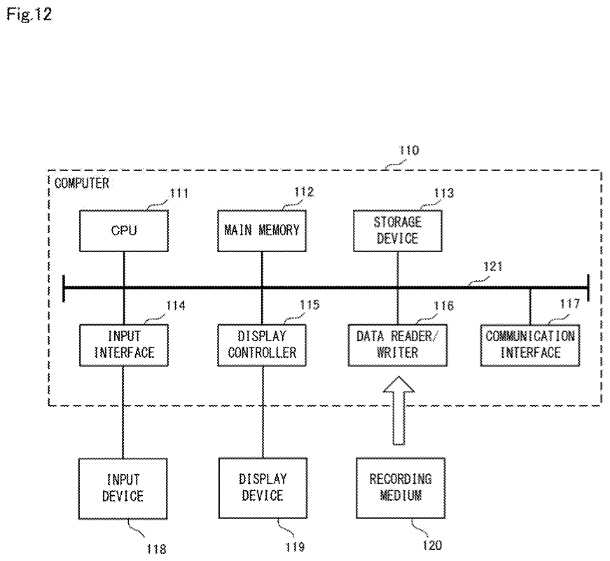

Here, a computer that realizes the control device of an odor detection apparatus by executing programs of the first and second example embodiments will be described using FIG. 12. FIG. 12 is a block diagram showing an example of a computer that realizes the odor detection apparatus in the first and second example embodiments of the invention.

As shown in FIG. 12, a computer 110 includes a CPU 111, a main memory 112, a storage device 113, an input interface 114, a display controller 115, a data reader/writer 116, and a communication interface 117. These constituent elements are connected in a manner that enables data communication, via a bus 121. Also, the computer 110 may include a GPU (Graphics Processing Unit) or an FPGA (Field-Programmable Gate Array), in addition to the CPU 111 or instead of the CPU 111.

The CPU 111 implements various computational operations, by extracting a program (codes) of the example embodiments stored in the storage device 113 to the main memory 112, and executing these codes in predetermined order. The main memory 112, typically, is a volatile storage device such as a DRAM (Dynamic Random Access Memory). Also, programs in the first and second example embodiments may be provided in a state of being stored on a computer-readable recording medium 120, or may be sent from a network connected via the communication interface 117.

Also, a semiconductor storage device such as a flash memory is given as a specific example of the storage device 113, other than a hard disk drive. The input interface 114 mediates data transmission between the CPU 111 and input devices 118 such as a keyboard and a mouse. The display controller 115 is connected to a display device 119 and controls display by the display device 119.

The data reader/writer 116 mediates data transmission between the CPU 111 and the recording medium 120, and executes readout of programs from the recording medium 120 and writing of processing results of the computer 110 to the recording medium 120. The communication interface 117 mediates data transmission between the CPU 111 and other computers.

Also, a general-purpose semiconductor storage device such as a CF (Compact Flash (registered trademark)) card or an SD (Secure Digital) card, a magnetic recording medium such as a flexible disk, and an optical recording medium such as a CD-ROM (Compact Disk Read Only Memory) are given as specific examples of the recording medium 120.

Note that the odor detection apparatus in the example embodiments is also realizable by using hardware corresponding to the respective constituent elements, rather than by a computer on which programs are installed. Furthermore, the odor detection apparatus may be realized in part by programs, and the remaining portion may be realized by hardware.

The example embodiments described above can be partially or wholly realized by supplementary notes 1 to 12 described below, although the invention is not limited to the following description.

### Supplementary Note 1

An odor sensing apparatus including:

a first odor sensor provided with a sensitive membrane;

a second odor sensor provided with an identical sensitive membrane to the sensitive membrane of the first odor sensor; and

a control device,

wherein the control device includes:

a sensor data acquisition unit configured to acquire first sensor data output by the first odor sensor and second sensor data output by the second odor sensor;

a calculation processing unit configured to calculate a difference between the first sensor data and the second sensor data; and

a determination unit configured to determine, when the sensitive membrane of one of the odor sensors is in a steady state, whether the sensitive membrane of the other odor sensor is in a steady state, based on the difference.

### Supplementary Note 2

The odor detection apparatus according to supplementary note 1,

wherein the control device further includes an odor detection unit configured to detect an odor, based on the sensor data from one of the first odor sensor and the second odor sensor.

### Supplementary Note 3

The odor detection apparatus according to supplementary note 2,

wherein the first odor sensor and the second odor sensor are odor sensors configured to output sensor data in reaction to a plurality of types of odors.

### Supplementary Note 4

The odor detection apparatus according to supplementary note 2 or 3, further including:

a first chamber in which the first odor sensor is disposed;

a second chamber in which the second odor sensor is disposed;

a sample gas supply unit configured to supply a sample gas containing odor molecules to the first chamber and the second chamber; and

a purge gas supply unit configured to supply a purge gas for eliminating the odor molecules from the sensitive membrane to the first chamber and the second chamber,

wherein the control device, after supply of the sample gas by the sample gas supply unit has ended, performs acquisition of the first sensor data and the second sensor data by the sensor data acquisition unit, calculation of the difference by the calculation processing unit, and determination by the determination unit, while causing the purge gas supply unit to supply the purge gas to the first chamber and the second chamber.

### Supplementary Note 5

An odor detection method for detecting an odor, using a first odor sensor provided with a sensitive membrane and a second odor sensor provided with an identical sensitive membrane to the sensitive membrane of the first odor sensor, the method including:

(a) a step of acquiring first sensor data output by the first odor sensor and second sensor data output by the second odor sensor;

(b) a step of calculating a difference between the first sensor data and the second sensor data; and

(c) a step of determining, when the sensitive membrane of one of the odor sensors is in a steady state, whether the sensitive membrane of the other odor sensor is in a steady state, based on the difference.

### Supplementary Note 6

The odor detection method according to supplementary note 5, further comprising:

(d) a step of detecting an odor, based on the sensor data from one of the first odor sensor and the second odor sensor.

### Supplementary Note 7

The odor detection method according to supplementary note 6,

wherein the first odor sensor and the second odor sensor are odor sensors configured to output sensor data in reaction to a plurality of types of odors.

### Supplementary Note 8

The odor detection method according to supplementary note 6 or 7, further using:

a first chamber in which the first odor sensor is disposed;

a second chamber in which the second odor sensor is disposed;

a sample gas supply unit configured to supply a sample gas containing odor molecules to the first chamber and the second chamber; and

a purge gas supply unit configured to supply a purge gas for eliminating the odor molecules from the sensitive membrane to the first chamber and the second chamber,

wherein, in the (d) step, the sample gas is supplied by the sample gas supply unit, and, after supply of the sample gas has ended, acquisition of the first sensor data and the second sensor data in the (a) step, calculation of the difference in the (b) step, and determination in the (c) step are performed, while causing the purge gas supply unit to supply the purge gas to the first chamber and the second chamber.

### Supplementary Note 9

A computer-readable recording medium that includes a program recorded thereon, in an odor sensing apparatus including a first odor sensor provided with a sensitive membrane, a second odor sensor provided with an identical sensitive membrane to the sensitive membrane of the first odor sensor and a computer, the program including instructions that cause the computer to carry out:

(a) a step of acquiring first sensor data output by the first odor sensor and second sensor data output by the second odor sensor;

(b) a step of calculating a difference between the first sensor data and the second sensor data; and

(c) a step of determining, when the sensitive membrane of one of the odor sensors is in a steady state, whether the sensitive membrane of the other odor sensor is in a steady state, based on the difference.

### Supplementary Note 10

The computer-readable recording medium according to supplementary note 9, the program further including instructions that cause the computer to carry out:

(d) a step of detecting an odor, based on the sensor data from one of the first odor sensor and the second odor sensor.

### Supplementary Note 11

The computer-readable recording medium according to supplementary note 10, wherein the first odor sensor and the second odor sensor are odor sensors configured to output sensor data in reaction to a plurality of types of odors.

### Supplementary Note 12

The computer-readable recording medium according to supplementary note 10 or 11,

wherein the odor sensing apparatus further includes a first chamber in which the first odor sensor is disposed, a second chamber in which the second odor sensor is disposed, a sample gas supply unit configured to supply a sample gas containing odor molecules to the first chamber and the second chamber, and a purge gas supply unit configured to supply a purge gas for eliminating the odor molecules from the sensitive membrane to the first chamber and the second chamber, and

in the (d) step, the program causes the computer to supply the sample gas with the sample gas supply unit, and, after supply of the sample gas has ended, causes the computer to perform acquisition of the first sensor data and the second sensor data in the (a) step, calculation of the difference in the (b) step, and determination in the (c) step, while performing supply of the purge gas to the first chamber and the second chamber by the purge gas supply unit.

Although the invention of the present application has been described above with reference to example embodiments, the invention is not limited to the foregoing example embodiments. Various modifications apparent to those skilled in the art can be made to the configurations and details of the invention of the present application within the scope of the invention.

This application is based upon and claims the benefit of priority from Japanese application No. 2018-032076, filed on Feb. 26, 2018, the disclosure of which is incorporated herein in its entirety by reference.

### INDUSTRIAL APPLICABILITY

As described above, according to the invention, it can be determined whether a sensitive membrane is in a steady state in elimination processing of an odor sensor. The invention is useful in fields in which odor sensors are utilized.

### LIST OF REFERENCE SIGNS

10 First odor sensor

20 Second odor sensor

30 Control device

31 Sensor data acquisition unit

32 Calculation processing unit

33 Determination unit

34 Odor detection unit

40 Display device

50, 60 Chamber

51 Fruit

52 Building

53 Horse

70 First chamber

71, 72 Inlet port

73 Exhaust port

80 Second chamber

81 Inlet port

82 Exhaust port

90 Sample gas supply unit

91 Purge gas supply unit

100 Odor detection apparatus (first example embodiment)

101 Casing

110 Computer

111 CPU

112 Main memory

113 Storage device

114 Input interface

115 Display controller

116 Data reader/writer

117 Communication interface

118 Input device

119 Display device

120 Recording medium

121 Bus

200 Odor detection apparatus (second example embodiment)

1. An odor sensing apparatus comprising:
a first odor sensor provided with a sensitive membrane;
a second odor sensor provided with an identical sensitive membrane to the sensitive membrane of the first odor sensor; and
a control device,
wherein the control device includes:
a sensor data acquisition unit configured to acquire first sensor data output by the first odor sensor and second sensor data output by the second odor sensor;
a calculation processing unit configured to calculate a difference between the first sensor data and the second sensor data; and
a determination unit configured to determine, when the sensitive membrane of one of the odor sensors is in a steady state, whether the sensitive membrane of the other odor sensor is in a steady state, based on the difference.

2. The odor detection apparatus according to claim 1,
wherein the control device further includes an odor detection unit configured to detect an odor, based on the sensor data from one of the first odor sensor and the second odor sensor.

3. The odor detection apparatus according to claim 2,
wherein the first odor sensor and the second odor sensor are odor sensors configured to output sensor data in reaction to a plurality of types of odors.

4. The odor detection apparatus according to claim 2, further comprising:
a first chamber in which the first odor sensor is disposed;
a second chamber in which the second odor sensor is disposed;
a sample gas supply unit configured to supply a sample gas containing odor molecules to the first chamber and the second chamber; and
a purge gas supply unit configured to supply a purge gas for eliminating the odor molecules from the sensitive membrane to the first chamber and the second chamber,
wherein the control device, after supply of the sample gas by the sample gas supply unit has ended, performs acquisition of the first sensor data and the second sensor data by the sensor data acquisition unit, calculation of the difference by the calculation processing unit, and determination by the determination unit, while causing the purge gas supply unit to supply the purge gas to the first chamber and the second chamber.

5. An odor detection method for detecting an odor, using a first odor sensor provided with a sensitive membrane and a second odor sensor provided with an identical sensitive membrane to the sensitive membrane of the first odor sensor, the method comprising:
acquiring first sensor data output by the first odor sensor and second sensor data output by the second odor sensor;
calculating a difference between the first sensor data and the second sensor data; and
determining, when the sensitive membrane of one of the odor sensors is in a steady state, whether the sensitive membrane of the other odor sensor is in a steady state, based on the difference.

6. The odor detection method according to claim 5, further comprising:
detecting an odor, based on the sensor data from one of the first odor sensor and the second odor sensor.

7. The odor detection method according to claim 6,
wherein the first odor sensor and the second odor sensor are odor sensors configured to output sensor data in reaction to a plurality of types of odors.

8. The odor detection method according to claim 6, wherein the method uses:
a first chamber in which the first odor sensor is disposed;
a second chamber in which the second odor sensor is disposed;
a sample gas supply unit configured to supply a sample gas containing odor molecules to the first chamber and the second chamber; and
a purge gas supply unit configured to supply a purge gas for eliminating the odor molecules from the sensitive membrane to the first chamber and the second chamber,
and wherein, in the detecting the odor, the sample gas is supplied by the sample gas supply unit, and, after supply of the sample gas has ended, acquisition of the first sensor data and the second sensor data, calculation of the difference, and determination are performed, while causing the purge gas supply unit to supply the purge gas to the first chamber and the second chamber.

9. A non-transitory computer-readable recording medium that includes a program recorded thereon, in an odor sensing apparatus including a first odor sensor provided with a sensitive membrane, a second odor sensor provided with an identical sensitive membrane to the sensitive membrane of the first odor sensor and a computer, the program including instructions that cause the computer to execute:
acquiring first sensor data output by the first odor sensor and second sensor data output by the second odor sensor;
calculating a difference between the first sensor data and the second sensor data; and
determining, when the sensitive membrane of one of the odor sensors is in a steady state, whether the sensitive membrane of the other odor sensor is in a steady state, based on the difference.

10. The non-transitory computer-readable recording medium according to claim 9, the program further including instructions that cause the computer to carry out:
detecting an odor, based on the sensor data from one of the first odor sensor and the second odor sensor.

11. The non-transitory computer-readable recording medium according to claim 10,
wherein the first odor sensor and the second odor sensor are odor sensors configured to output sensor data in reaction to a plurality of types of odors.

12. The non-transitory computer-readable recording medium according to claim 10,
wherein the odor sensing apparatus further includes a first chamber in which the first odor sensor is disposed, a second chamber in which the second odor sensor is disposed, a sample gas supply unit configured to supply a sample gas containing odor molecules to the first chamber and the second chamber, and a purge gas supply unit configured to supply a purge gas for eliminating the odor molecules from the sensitive membrane to the first chamber and the second chamber, and
in the detecting the odor, the program causes the computer to supply the sample gas with the sample gas supply unit, and, after supply of the sample gas has ended, causes the computer to perform acquisition of the first sensor data and the second sensor data, calculation of the difference, and determination, while performing supply of the purge gas to the first chamber and the second chamber by the purge gas supply unit.
